(19) United States
(12) Patent Application Publication
(10) Pub. No.: US 2014/0207103 A1
(43) Pub. Date: Jul. 24, 2014

(54) HAND-HELD ADIPOSE PROCESSOR AND CELL CONCENTRATOR

(71) Applicant: Tissue Genesis, Inc., Honolulu, HI (US)

(72) Inventors: Rolf Wolters, Kailua, HI (US); Anthony Yang, Honolulu, HI (US); Josh Nelson, Kaneohe, HI (US); Stuart K. Williams, Harrods Creek, KY (US)

(73) Assignee: Tissue Genesis, Inc., Honolulu, HI (US)

(21) Appl. No.: 14/221,272

(22) Filed: Mar. 20, 2014

Related U.S. Application Data
(63) Continuation-in-part of application No. 13/844,548, filed on Mar. 15, 2013.
(60) Provisional application No. 61/703,742, filed on Sep. 20, 2012.

(51) Int. Cl. A61M 1/00; G01N 1/40

(52) U.S. Cl. CPC A61M 1/0056; G01N 1/4044
USPC 604/506; 435/309.1; 604/542

(57) ABSTRACT

Devices and methods are provided for processing adipose tissue with a hand-held device. This device may include a processing chamber, a cannula, a vacuum source, a digestion area, and a product cell concentration chamber.

### CROSS-REFERENCE TO RELATED APPLICATION

This application is a continuation-in-part of U.S. patent application Ser. No. 13/844,548, filed Mar. 15, 2013, which claims the benefit of provisional patent application Ser. No. 61/703,742, filed Sep. 20, 2012, titled “Hand-Held Micro-Liposuction Adipose Harvester, Processor, and Cell Concentrator”, the contents which are incorporated herein by reference in their entirety.

### TECHNICAL FIELD

This disclosure relates to the processing of adipose tissue, the purification, and the subsequent concentration of product resulting from said processing in an integrated system.

### BACKGROUND

Adipose tissue has numerous uses. When such tissue is digested, the freed cells are capable of a multitude of uses, including tissue engineering, tissue repair, release of therapeutic factors by the cell, including factors released as a result of genetic engineering of the cells.

A problem with existing methods of extracting and isolating adipose cells is that existing equipment, such as liposuction cannulas and pumps and digestion devices, is not expected to be very portable. Furthermore, the liposuction, digestion, and cell concentration are often carried out in separate devices, and there are consequent issues in maintaining sterility as tissue and cells are transferred between devices. Consequently, it would be advantageous to have a portable, sterile device which was capable of carrying out the functions of extracting tissue, digesting the tissue, and concentrating the cells.

### BRIEF SUMMARY

Described herein are various inventions, particular examples of which are summarized here. In one embodiment, there is described a hand-held device for processing and concentrating a SVF and cell product. This device may comprise: a processing chamber comprising a digestion area; a filter within the processing chamber, which may be configured to allow SVF and liquids to pass from the processing chamber through the filter, while not allowing larger solid materials to pass through the filter; a wash chamber in which the SVF and cell product may be contained by a porous barrier that enables active fluid transfer; and a SVF and cell product concentrator.

In another embodiment, a method is described for processing and concentrating a SVF and cell product. This method may comprise: providing a hand-held device comprising: a processing chamber comprising a digestion area; a cannula with one end connected to the processing chamber in an airtight manner, and the other end open to the atmosphere; and a vacuum source connected to the processing chamber such that when the cannula is inserted into adipose tissue and the vacuum source is activated, the adipose tissue can be aspirated through the cannula into the processing chamber; inserting a tip of the cannula into adipose tissue within a patient; opening the valve to induce a vacuum within the chamber, which may cause a portion of the adipose tissue to move through the cannula into the processing chamber; situating the adipose tissue within the digestion area; digesting the portion of the adipose tissue in the chamber by incubation in the presence of a digesting enzyme, while maintaining a constant temperature; and passing the incubated adipose tissue product through a filter within the processing chamber, such that product SVF and liquids pass from the processing chamber into wash chamber.

Various additional embodiments, including additions and modifications to the above embodiments, are described herein.

### BRIEF DESCRIPTION OF THE DRAWINGS

The accompanying drawings, which are incorporated into this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within this field.

In the drawings:

FIG. 1 is an illustration of a hand-held cartridge hybrid system.

FIG. 2 is an illustration of an alternate embodiment of a hand-held cartridge system.

FIG. 3 is an illustration of a hand-held cartridge system with a pre-processor.

FIG. 4 is an illustration of an all-in-one hand-held device for lipoaspiration and processing.

FIG. 5 is a flow chart illustrating an example of liposuction and fully automatic processing.

FIG. 6 is a flow chart illustrating an example of pre-processing and fully automatic processing.

### DETAILED DESCRIPTION

Various embodiments of the present inventions are described herein in the context of concentrating purified cell aggregates. The following detailed description is illustrative only and is not intended to be in any way limiting. Other embodiments will readily suggest themselves to skilled people within this field having the benefit of this disclosure.

In the interest of clarity, not all of the routine features of the implementations described herein are shown and described. In the development of any such actual implementation, numerous implementation-specific decisions must be made in order to achieve the developer's specific goals, such as compliance with application, safety, regulatory, and business constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Described herein are examples of a set of related compact devices that isolate cells from adipose tissue, such as stromal vascular fraction (SVF), and including stem cells. One embodiment is a device that may process, purify, and concentrate cell product. In one embodiment, it may also harvest fat. Adipose tissue may, in one example, be processed in one continuous cycle.

One embodiment is an integrated system that is miniaturized and may be hand-held. Use of the term hand-held herein means a portable device that is capable of being held in one hand. A possible volume of adipose tissue harvested may be less than approximately 20 ml. Larger sizes of adipose tissue (e.g., 30 ml, 40 ml, 100 ml., etc.) may be also be accommodated by scaling the device. In one example, the device may accept aspirated adipose tissue into a cassette style configuration that comprises a series of connected syringe bodies. Each syringe body may contribute a step to an overall process of digestion and processing, which may collectively enable the process in a continuous and closed cycle.

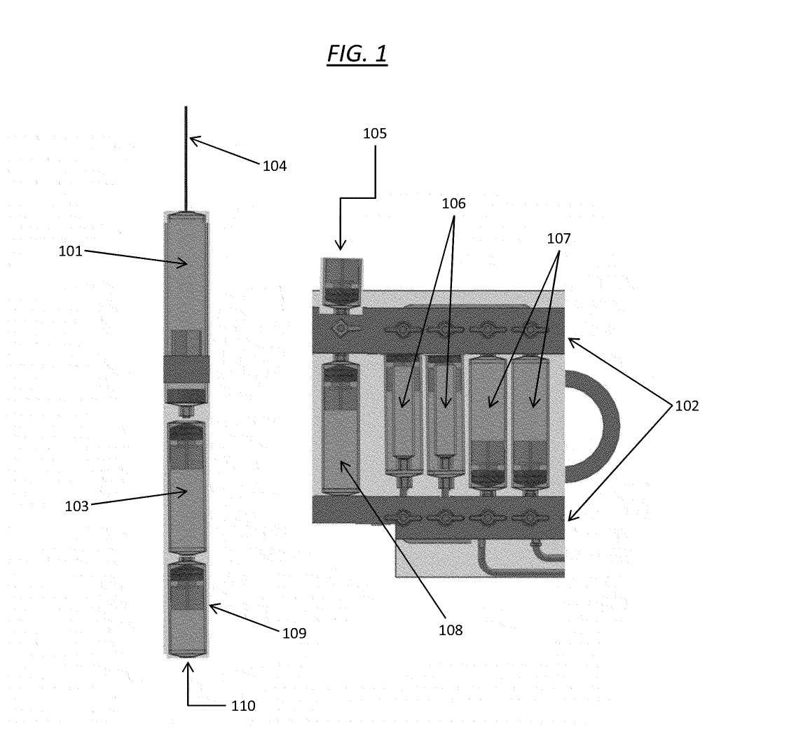

FIG. 1 illustrates one embodiment of such a device in the form of a hand-held cartridge hybrid. A cannula 104 may be used for liposuction to extract adipose tissue from a patient. A vacuum may be supplied, for example, by an external vacuum pump or by a vacuum aspiration chamber 103. In one embodiment, a backup vacuum chamber 109 may be connected as an extra vacuum source. For example, this backup chamber may be used if the vacuum in the primary chamber is lost during use. Vacuum chambers 103, 109 may be recharged through a vacuum recharge access 110. Such recharging may, for example, take place during the liposuction procedure if for some reason the vacuum is lost.

The vacuum may be used to create adequate negative pressure through the cannula 104 such that adipose tissue with which the cannula comes in contact may be forcibly removed from a host and collected by the device. Solid tissue may be collected in a chamber 101, which may also serve as a digestion chamber. Aspirant from the liposuction procedure may be collected into the vacuum aspiration chamber 103. Fluidics control valves are shown as 102.

If vacuum is provided by one or more connected evacuated vessels (e.g., 103, 109), the vessel(s) should be of sufficient volume to provide necessary suction for liposuction. In other embodiments, a vacuum pump may be attached to the device during operation. In another alternative, a vacuum pump in a base unit may serve to evacuate one or more vessels in the device while it is cradled in the base. Thus, the hand-held device may be pumped down so that the device can operate independently of the base, providing vacuum for the lipoaspiration procedure. In one example, a vacuum may be engaged by a push of a button on the handle by the operator, and the vacuum may be disengaged upon the release of said button.

Associated with and/or connected to the device may be a pressure pump or vessel able to provide a supply gas, for example compressed air, oxygen or nitrogen, and a vacuum pump able to provide negative pressure. This positive or negative pressure may be used to move fluids throughout the system. An alternate embodiment produces positive pressure through the use of an incorporated micro-pump or series of micro-pumps. In another embodiment, a pump in a base may pressurize a vessel in the hand-held while it is cradled in a base, so that the hand-held may operate independent of the base. In another embodiment, a pump in the base may supply positive pressure and negative pressure for the hand-held only while it is cradled, thereby only moving fluids by pressure during the cradled phase. Fluid flow may be managed by the cycling of valves, including check valves, electrically actuated valves, and other valves.

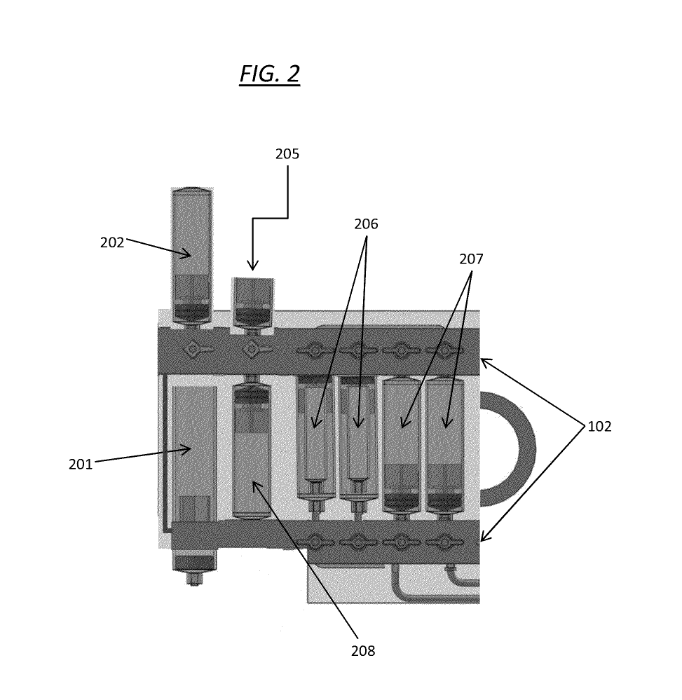

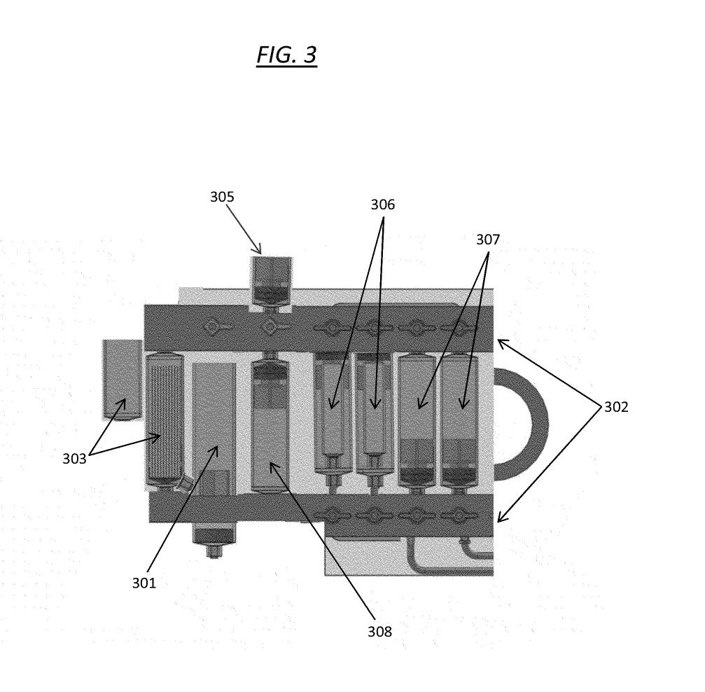

FIG. 1 also shows an example hand-held cartridge unit (right) associated with the device shown on the left. It may contain a port for an enzyme vial 105, a trituration syringe or digestate syringe 108, a filter or series of filters 106, and wash media syringes 107. Various other configurations of syringe, hand-held device, base-station, or cartridge unit are possible, and some of them are also illustrated in FIG. 2 and FIG. 3. For example, a cartridge/base station configuration may permit a cartridge containing adipose cells from liposuction to slide into the receiver-slot of the small sized base station. An alternate embodiment may involve a combination of a hand-held device and a cartridge that may be coupled and inserted into a base station, decoupling the hand-held during a lipoaspiration step. An alternate embodiment is a single-piece hand-held unit and a base. An alternate embodiment may involve multiple hand-held units in a carousel-style base, thereby giving an operator the option to harvest and process incremental volumes of fat, depending on their intended use. In another alternate embodiment, the carousel base may double as a centrifuge.

FIG. 2 illustrates a related configuration. In addition to the enzyme vial 205, filter or series of filters 206, wash media syringes 207, and trituration syringe/digestate syringe 208, there is a port for attachment of a digestion chamber 201 and adipose syringe 202. FIG. 3 also shows enzyme vial 305, filter or series of filters 306, wash media syringes 307, and trituration syringe/digestate syringe 308, and digestion chamber 301, but also illustrates an adipose mincer/preprocessor 303. Appropriate sterile, physical connections including pumps and/or valves may be routed between the various cartridges, so that, for example, wash from wash media syringes 207 may flow to digestion chamber 201.

After collection in the digestion chamber 101 (or 201, 301), tissue may be processed by enzymatic digestion under the appropriate conditions. For example, the tissue may be digested with collagenase at a physiological temperature, for an amount of time sufficient to break down the interstitial matrix of the tissue. Other means of digesting adipose tissue are known in the art. Thereafter, the suspension may be purified and concentrated, leaving behind concentrated cells, as described below.

In one embodiment, processing of the collected adipose tissue may involve mechanically mincing the tissue in a mincer 303, thereby preparing the tissue to undergo further processing via enzymatic digestion. The tissue may then be washed and concentrated by removing fluid and waste.

Enzymes may be supplied in specified aseptic vials 105, 205, 305 (supply vials). These vials may in one embodiment be introduced into the device by “snapping-in” the vial in a specified location, during which an incorporated vial access spike punctures the seal. This vial may then remain within the device for the duration of the process.

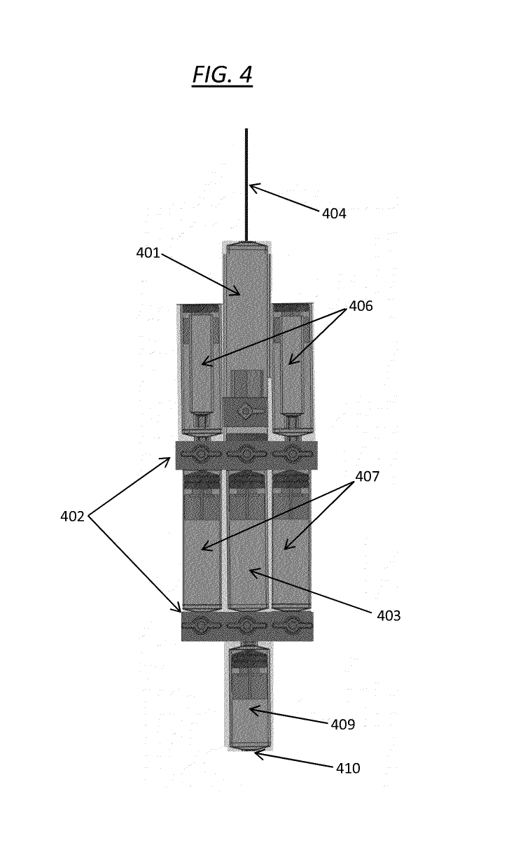

FIG. 4 illustrates a configuration in which all steps are integrated into a single assembly. A cannula 404 may be used for liposuction to extract adipose tissue from a patient. A vacuum may be supplied by a vacuum aspiration chamber 403, with a secondary reserve vacuum chamber 409. These chambers can be accessed for initial evacuation or for a re-charge via an interface 410. After lipoaspiration is complete, gravity decanted fluid can be drained into 403, leaving adipose tissue ready for enzymatic digestion in 401. Upon completion, successive steps of filtration 406 using wash fluid from 407 may complete the processing.

In one embodiment, the device may comprise durable components and disposable components. In particular, any component that comes in contact with fluid may be disposable. In one embodiment, the entire hand-held may be a disposable, into which all fluid-contacting components necessary for the process may be incorporated. In one embodiment, the handle of the hand-held device may also include a connector onto which a cannula may be mounted. Valves that may control fluid path flow may be coupled to and actuated by the base

If the device has a base, the base may contain the heater, pressure and vacuum pumps, and a physical coupling with the cartridge which may be inserted in the base. Adipose tissue may then be introduced into the cartridge for processing.

The use of the device may in one embodiment involve three steps: aspiration, processing, and use of product. Aspiration is envisaged to be minimally invasive due to the nature of small volume “micro-liposuction.” The hand-held design may be intended to be small and easy to handle during the aspiration step. Upon completion of aspiration, the hand-held may in one embodiment be inserted into the base. The base may provide heat to the processor region of the hand-held to optimize the digestive process for a specified amount of time. An alternate embodiment includes an inductive charger to recharge a battery in the hand-held, so that the battery may provide electricity for a heater. In an alternate embodiment, the base may contain a heater, wash media, pressure and vacuum pumps, or any combination thereof, coupling with the hand-held when it is cradled in the base.

In one example, a device may use a combination of flow-through filters and containment filters. Larger pore size filters to trap large materials and pass the desired product via positive pressure may be employed, while subsequent filters that prevents product from escaping via containment follow sequentially. In one embodiment, digestate may be washed via a dynamic processes in which it is introduced into a submersed containment area, flushed with wash media, and retrieved for further processing. The containment area may comprise a tubular filter with pore sizes small enough to prevent stem cells from passing, but large enough to let non-nucleated cells (red blood cells) and cell fragments to pass. An alternate containment area may be a porous substrate that prevents stem cells from passing, but allows residual enzyme and endotoxins to wash away.

Cells may in one example be concentrated and purified using a hollow-fiber bioreactor or porous substrate tube. As the medium passes through the tubes, fluid and enzymes may diffuse out of the fiber for disposal, while cells and stromal and vascular fractions may pass through as retentate. This process may be repeated until the desired concentration of cells is achieved. Additional wash can be added to arrive at a preset dilution.

When processing is complete, product cells may be extracted via syringe, ready for use. In an alternate embodiment, the cell product may be delivered by the device through a dedicated needle, at a specified delivery pressure and concentration. In another alternate embodiment, the cell product may be extracted from the hand-held after processing via a syringe that contains an intermediate filter assembly. The negative pressure from withdrawing the syringe-plunger may pull the cells through the filter assembly before entering the syringe.

In one example, a device may use positive flow-through filters and negative containment filters. Such a device may use a dynamic wash process to purify the product. Such a device may also use a hollow-fiber filter, a porous substrate or a porous tube to concentrate the product. Advantages of the above inventions include the speed by which the device automatically processes small volumes of fat. This has significant appeal by minimizing the impact on the clinical work-flow. Due to the small volume minimum processing requirements, fat-harvesting may occur from multiple areas on the body of the patient (using multiple devices), or allowing for periodic harvest over time, always being able to access virgin fat. Furthermore, dialing in the amount of fat needed and processed depending on stem cell application is appealing (take only what you need).

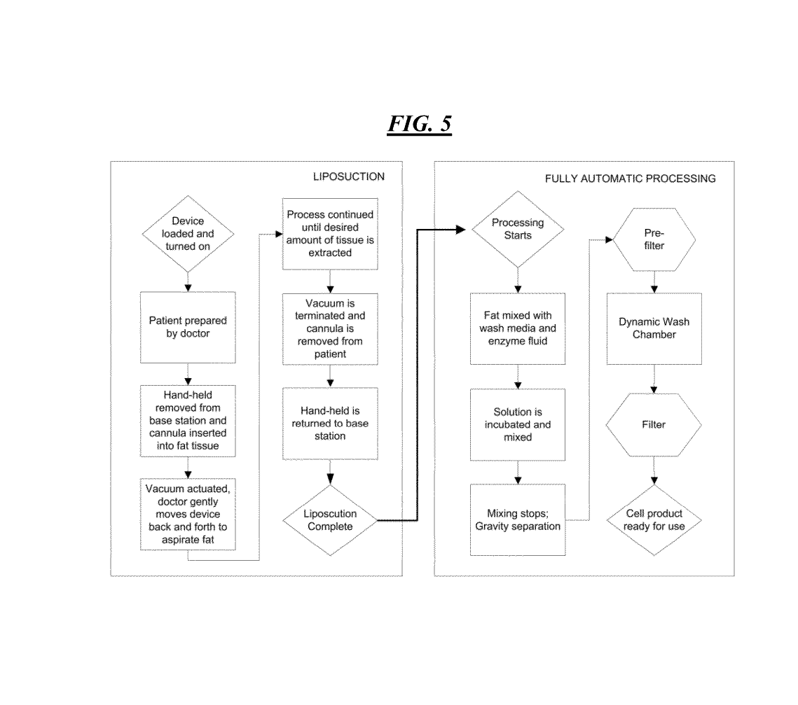

FIG. 5 is a flow chart showing an example of the steps one might take in using liposuction in accordance with the devices described herein, followed by a fully-automatic processing. In this example, a hand-held device, with a vacuum, may be used to aspirate adipose tissue during a liposuction procedure. During processing, fat may be mixed with wash media and enzyme fluid. The solution is incubated and mixed, then the mixing may be stopped while gravity is allowed to decant the fluid, separating oils from the cell containing suspension. The material passes through a pre-filter, and then is subject to a dynamic wash, after which may pass through a filter, such as a membrane based tangential flow filtration (TFF). At this point, the cells may be ready for use.

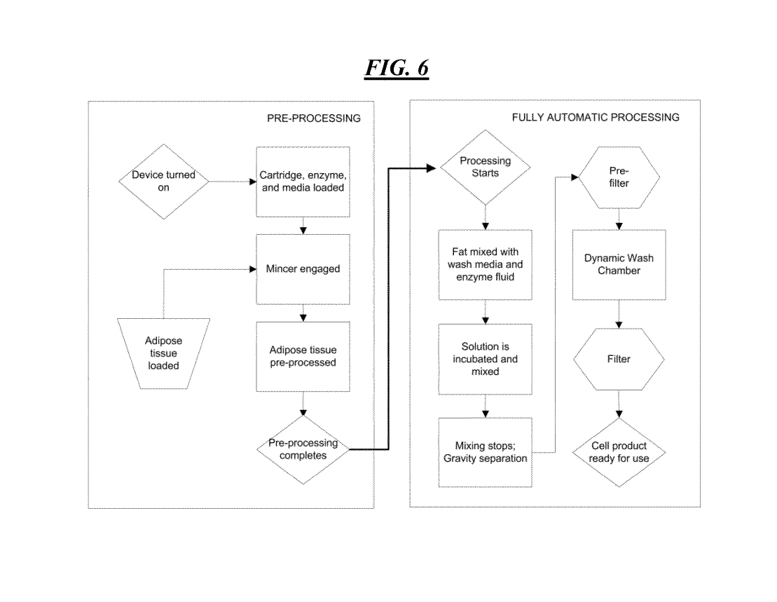

FIG. 6 describes a related process, except that a pre-processing step is added, and the liposuction step is not shown. Here, adipose tissue may be loaded into the device and passed through a mincer and then pre-processed.

### Further Example

What follows is an illustrative example of a method of using one or more of the devices described herein.

Step 1: 15 cc's of decanted aspirated fat is injected into device.

Step 2: Device pre-processes fat to mince connective tissue.

Step 3: Device automatically initiates the digestion process.

Step 4: Temperature is raised to around 37 C for digestion.

Step 5: Digestate is washed to arrest digestion and to remove red blood cells

Step 6: Cells are automatically concentrated via filtration.

Step 7: The doctor attaches a syringe to the product access point to retrieve final product. In an alternative embodiment, the doctor retrieves a separate product reservoir from the device to access the final product.

What is claimed is:

1. A hand-held device for processing and concentrating a SVF and cell product, comprising:
a processing chamber comprising a digestion area;
a filter within the processing chamber, configured to allow SVF and liquids to pass from the processing chamber through the filter, while not allowing larger solid materials to pass through the filter;
a wash chamber in which the SVF and cell product are contained by a porous barrier that enables active fluid transfer; and
a SVF and cell product concentrator.

2. The device of claim 1, further comprising a pre-processing module with a mincer, whereby tissue may be minced such that connective tissue strands within the tissue are cut.

3. The device of claim 1, comprises a heater for heating a portion of the portable unit that includes the digestion area.

4. The device of claim 1, wherein the hand-held device comprises a hand-held unit and a hand-held cartridge unit; wherein the cartridge unit comprises:
a product cell concentration chamber capable of separating liquids and subcellular sized components from cells and cell-sized and larger structures; and
a plurality of fluidics control valves configured to allow passage of liquids and/or entrained solids between multiple cartridges connected to the cartridge unit.

5. The device of claim 4, further comprising:
a cannula with one end connected to the processing chamber in an airtight manner, and the other end open to the atmosphere;
a vacuum source connected directly or indirectly to the processing chamber such that when the cannula is inserted into adipose tissue and the vacuum source is activated, the adipose tissue can be aspirated through the cannula into the processing chamber;
a port for establishing a fluid connection with the cartridge unit.

6. The device of claim 5, further comprising a second vacuum source connectable by a valve to the processing chamber.

7. The device of claim 4, further comprising:
a heater for heating a portion of the portable unit that includes the digestion area;
a hand-held base adapted to hold the cartridge unit in a position suitable for digestion of adipose tissue;
circuitry for controlling the temperature of the heater; and
circuitry for controlling the fluidics control valves.

8. The device of claim 7, wherein a hand-held device attaches to the base and, in its attached configuration, is in fluid communication with the cartridge unit.

9. The device of claim 1, further comprising:
a cannula with one end connected to the processing chamber in an airtight manner, and the other end open to the atmosphere; and
a vacuum source connected directly or indirectly to the processing chamber such that when the cannula is inserted into adipose tissue and the vacuum source is activated, the adipose tissue can be aspirated through the cannula into the processing chamber.

10. A plurality of devices of claim 1, further comprising a base adapted to hold the plurality of devices in a position suitable for digestion of adipose tissue, wherein the base is a rotating carousel.

11. A method for processing and concentrating a SVF and cell product, comprising:
providing a hand-held device comprising:
a processing chamber comprising a digestion area;
a cannula with one end connected to the processing chamber in an airtight manner, and the other end open to the atmosphere; and
a vacuum source connected to the processing chamber such that when the cannula is inserted into adipose tissue and the vacuum source is activated, the adipose tissue can be aspirated through the cannula into the processing chamber;
inserting a tip of the cannula into adipose tissue within a patient;
opening the valve to induce a vacuum within the chamber, thereby causing a portion of the adipose tissue to move through the cannula into the processing chamber;
situating the adipose tissue within the digestion area;
digesting the portion of the adipose tissue in the chamber by incubation in the presence of a digesting enzyme, while maintaining a constant temperature;
passing the incubated adipose tissue product through a filter within the processing chamber, such that product SVF and liquids pass from the processing chamber into wash chamber.

12. The method of claim 11, further comprising passing the adipose tissue through a mincer.

13. The method of claim 11, further comprising subjecting the SVF to tangential flow filtration.
